(19) United States
(12) Patent Application Publication
(10) Pub. No.: US 2021/0388086 A1
(43) Pub. Date: Dec. 16, 2021

(54) ANTIBODIES FOR DETECTING VARIOUS TUMOR CELLS AND USES THEREOF

(71) Applicant: SHENZHEN UNIVERSITY, Shenzhen (CN)

(72) Inventors: Suping ZHANG, Shenzhen, Guangdong (CN); Thomas James KIPPS, Shenzhen, Guangdong (CN); Liufeng WU, Shenzhen, Guangdong (CN)

(21) Appl. No.: 17/369,988

(22) Filed: Jul. 8, 2021

Related U.S. Application Data
(63) Continuation of application No. PCT/CN2019/130486, filed on Dec. 31, 2019.

(30) Foreign Application Priority Data
Jan. 8, 2019 (CN) 201910015053.3

(51) Int. Cl. C07K 16/28; G01N 33/574; C12N 15/85; C07K 14/705; C07K 14/725

(52) U.S. Cl. CPC C07K 16/2803; G01N 33/57492; C12N 15/85; C07K 2319/03; C07K 14/70578; C07K 14/7051; C07K 2317/622; C07K 14/70517

(57) ABSTRACT

Disclosed are an anti-ROR2 antibody and a use thereof. The anti-ROR2 antibody includes: a light chain variable region with amino acid sequence shown in CSASSSVSYMHWYQ, IYDTSKLAS and CQQWSSNPPTFGAG, and a heavy chain variable region with amino acid sequence shown in YTITSYLMHWV, LEWIGYINPYNDGTKYNEKFKDKAT and CARSDVYYGVRFAYWGQG; a light chain variable region with amino acid sequence shown in CKASQNVGTNVAWFQ, IYLASYRYS and CQQYNSYPLTFGGG, and a heavy chain variable region with amino acid sequence shown in YTFTNYWIQWM, LEWIGEINPSNGRTDYNEKFKNRAT and CANYRPGYWGQG; and a light chain variable region with amino acid sequence shown in CSASSSISYMYWYQ, IYDTSILAS and CQQWSSYPFTFGSG, and a heavy chain variable region with amino acid sequence shown in YTFTSYLIHWV, LEWIGYINPYNDGTKYNEKFKDKAT and CARSDVYYGVRFAYWGQG.

### CROSS-REFERENCE TO RELATED APPLICATIONS

This application is a Continuation Application of International Application No. PCT/CN2019/130486, filed on Dec. 31, 2019, which claims priority to Chinese Application No. 201910015053.3, filed on Jan. 8, 2019, the entire disclosure of which is incorporated herein by reference.

This application contains a sequence listing submitted as an ASCII text file, named “Seq-List.txt” and created on Aug. 19, 2021, with 36 kilobytes in size. The material in the above-identified ASCII text file is incorporated herein by reference in its entirety.

### TECHNICAL FIELD

The present disclosure relates to the field of biomedicine, in particular to antibodies for detecting various tumor cells and uses thereof, and in particular to an anti-ROR2 antibody and a use thereof.

### BACKGROUND

The tyrosine kinase orphan-like receptor ROR2 is an important development-related regulatory protein, and ROR2 is highly expressed in various tissues during early embryonic development and plays an important role in tissue growth and differentiation. After the second trimester, the expression level of ROR2 gradually decreases. Except for expression in some osteoblasts and uterine cells, ROR2 is basically not expressed in adult tissues. The current research shows that ROR2 is highly expressed in many tumor tissues, including osteosarcoma, melanoma, colorectal cancer and gastric cancer, breast cancer, lymphoma, leukemia and other cancers. Therefore, many scholars believe that ROR2 is a disease-related gene and can be used as an ideal drug target.

ROR2 is a tyrosine kinase orphan receptor that is expressed in large quantities and plays an important role during embryonic development, and ROR2 is difficult to be detected in most adult tissues. Studies in recent years have found that although most normal tissues do not express ROR2, many malignant tumor cells including breast cancer, lung cancer, pancreatic cancer and other cells have high expression of ROR2. In addition, cancer cells with higher ROR2 expression levels are more malignant and prone to metastasis, recurrence, and poorer prognosis. ROR2 can usually be detected on tumor cells with a low degree of differentiation, which have strong recurrence and metastasis ability.

However, drug development for ROR2 needs to be improved.

### SUMMARY

The present disclosure aims to solve one of the technical problems in the related art to a certain extent. To this end, one purpose of the present disclosure is to provide an anti-ROR2 antibody and a use thereof.

The present disclosure is based on the inventor's following research findings: during the process of tumor research, a surface marker of tumor stem cells, such as CD133, CD44, CD24, ALDH1, or the like, is expressed in tumor cells and normal adult tissues. As a result, the anti-tumor stem cell drugs developed for these surface molecules will also cause certain toxicity to normal tissues. However, ROR2 is also abundantly expressed in tumor stem cells, but less expressed in normal cells. Therefore, the development of therapeutic antibodies against ROR2 will cause less damage to normal tissues. Thus, ROR2 is an ideal drug target. The drugs developed for ROR2 can be used in the treatment of tumors and cancer, and the damage to normal tissues will be relatively small.

To this end, the inventors of the present disclosure developed specific antibodies against ROR2 antigen through research. The antibodies provided in the present disclosure can bind to the ROR2 antigen with high specificity, so that the antibodies can be applied to the targeted therapy of tumors and the specific diagnosis and detection of the ROR2 antigen.

Specifically, the present disclosure provides the following technical solutions:

According to a first aspect of the present disclosure, the present disclosure provides an anti-ROR2 antibody or antigen-binding fragment, including at least one of the following: (1) a light chain variable region with amino acid sequence shown in CSASSSVSYMHWYQ, IYDTSKLAS and CQQWSSNPPTFGAG, and a heavy chain variable region with amino acid sequence shown in YTITSYLMHWV, LEWIGYINPYNDGTKYNEKFKDKAT and CARSDVYYGVRFAYWGQG; (2) a light chain variable region with amino acid sequence shown in CKASQNVGTNVAWFQ, IYLASYRYS and CQQYNSYPLTFGGG, and a heavy chain variable region with amino acid sequence shown in YTFTNYWIQWM, LEWIGEINPSNGRTDYNEKFKNRAT and CANYRPGYWGQG; (3) a light chain variable region with amino acid sequence shown in CSASSSISYMYWYQ, IYDTSILAS and CQQWSSYPFTFGSG, and a heavy chain variable region with amino acid sequence shown in YTFTSYLIHWV, LEWIGYINPYNDGTKYNEKFKDKAT and CARSDVYYGVRFAYWGQG; compared with (1) to (3), the antibody or antigen-binding fragment has an amino acid sequence with at least one conservative amino acid substitution. The antibody or antigen-binding fragment provided herein has high affinity with ROR2 antigen, exhibits affinity at a concentration of 0.0001 μg/ml, and can be used for a variety of ROR2 antigen-related detections. Moreover, it can be applied to the treatment of tumors and play a role in targeted therapy.

According to an embodiment of the present disclosure, the above-mentioned antibody or antigen-binding fragment further includes the following technical features.

In some embodiments of the present disclosure, the antibody or antigen-binding fragment includes at least one of the following: (a) a light chain variable region with amino acid sequence shown in SEQ ID NO: 1 and a heavy chain variable region with amino acid sequence shown in SEQ ID NO: 2; (b) a light chain variable region with amino acid sequence shown in SEQ ID NO: 3 and a heavy chain variable region with amino acid sequence shown in SEQ ID NO: 4; (c) a light chain variable region with amino acid sequence shown in SEQ ID NO: 5 and a heavy chain variable region with amino acid sequence shown in SEQ ID NO: 6; compared with (a)˜(c), the antibody or antigen-binding fragment has an amino acid sequence with at least one conservative amino acid substitution.

In some embodiments of the present disclosure, the antibody or antigen-binding fragment includes at least one of the following: a light chain with amino acid sequence shown in SEQ ID NO: 7 and a heavy chain with amino acid sequence shown in SEQ ID NO: 8; a light chain with amino acid sequence shown in SEQ ID NO: 9 and a heavy chain with amino acid sequence shown in SEQ ID NO: 10; and a light chain with amino acid sequence shown in SEQ ID NO: 11 and a heavy chain with amino acid sequence shown in SEQ ID NO: 12.

According to a second aspect of the present disclosure, the present disclosure provides an isolated polynucleotide encoding the antibody or antigen-binding fragment described in any one of the embodiments of the first aspect of the present disclosure.

According to an embodiment of the present disclosure, the polynucleotide includes at least one of the following:

a light chain variable region nucleotide sequence shown in SEQ ID NO: 13 and a heavy chain variable region nucleotide sequence shown in SEQ ID NO: 14;

a light chain variable region nucleotide sequence shown in SEQ ID NO: 15 and a heavy chain variable region nucleotide sequence shown in SEQ ID NO: 16;

a light chain variable region nucleotide sequence shown in SEQ ID NO: 17 and a heavy chain variable region nucleotide sequence shown in SEQ ID NO: 18;

a light chain nucleotide sequence shown in SEQ ID NO: 19 and a heavy chain nucleotide sequence shown in SEQ ID NO: 20;

a light chain nucleotide sequence shown in SEQ ID NO: 21 and a heavy chain nucleotide sequence shown in SEQ ID NO: 22;

a light chain nucleotide sequence shown in SEQ ID NO: 23 and a heavy chain nucleotide sequence shown in SEQ ID NO: 24;

compared with any light chain variable region nucleotide sequence described above, the polynucleotide has a sequence of more than 90% homology, optionally a sequence of more than 95% homology, preferably a sequence of more than 98% homology, more preferably a sequence of more than 99% homology;

compared with any heavy chain variable region nucleotide sequence described above, the polynucleotide has a sequence of more than 90% homology, optionally a sequence of more than 95% homology, preferably a sequence of more than 98% homology, more preferably a sequence of more than 99% homology;

compared with any light chain nucleotide sequence described above, the polynucleotide has a sequence of more than 90% homology, optionally a sequence of more than 95% homology, preferably a sequence of more than 98% homology, more preferably a sequence of more than 99% homology;

compared with any heavy chain nucleotide sequence described above, the polynucleotide has a sequence of more than 90% homology, optionally a sequence of more than 95% homology, preferably a sequence of more than 98% homology, more preferably a sequence of more than 99% homology.

According to a third aspect of the present disclosure, the present disclosure provides an expression vector including the polynucleotide described in any one of the embodiments of the second aspect of the present disclosure.

In some embodiments of the present disclosure, the expression vector further includes: a control element operably linked to the polynucleotide for controlling the expression of the polynucleotide in the host cell.

In some embodiments of the present disclosure, the control element includes at least one of the following: a promoter, an enhancer, and a terminator.

In some embodiments of the present disclosure, the host cell is a mammalian cell.

According to a fourth aspect of the present disclosure, the present disclosure provides a recombinant cell including the expression vector described in any one of the embodiments of the third aspect of the present disclosure.

According to a fifth aspect of the present disclosure, the present disclosure provides a method for preparing an anti-ROR2 antibody or antigen-binding fragment, including culturing the recombinant cell described in the fourth aspect of the present disclosure.

According to a sixth aspect of the present disclosure, the present disclosure provides a use of an antibody or antigen-binding fragment in the preparation of a medicine. The medicine is used to treat cancer, and the antibody or antigen-binding fragment is the antibody or antigen-binding fragment described in the first aspect of the present disclosure.

According to a seventh aspect of the present disclosure, the present disclosure provides a use of an antibody or antigen-binding fragment in the preparation of a kit. The kit is used for the diagnosis and detection of ROR2 antigen, and the antibody or antigen-binding fragment is the antibody or antigen-binding fragment described in the first aspect of the present disclosure.

In some embodiments of the present disclosure, the kit is used for immunoblotting, immunoprecipitation, and ELISA detection.

According to an eighth aspect of the present disclosure, the present disclosure provides a pharmaceutical composition, including: the antibody or antigen-binding fragment described in any one of the embodiments of the first aspect of the present disclosure and a pharmaceutically acceptable carrier.

According to a ninth aspect of the present disclosure, the present disclosure provides an anti-ROR2 chimeric antigen receptor, including: an extracellular domain, a transmembrane domain and an intracellular domain. The extracellular domain includes an antibody or antigen-binding fragment, the antibody or antigen-binding fragment is a single chain, and the antibody or antigen-binding fragment is the antibody or antigen-binding fragment according to any one of the embodiments of the first aspect of the present disclosure.

According to a tenth aspect of the present disclosure, the present disclosure provides a Car-T cell that expresses the anti-ROR2 chimeric antigen receptor described in the ninth aspect of the present disclosure.

### BRIEF DESCRIPTION OF THE DRAWINGS

FIG. 1 is an ELISA detection result diagram of Fab antibodies of A12, B22, and B30 at different concentrations according to an embodiment of the present disclosure.

FIG. 2 is an SDS-PAGE identification result diagram of different antibodies according to an embodiment of the present disclosure.

FIG. 3 is an ELISA detection result diagram of chimeric antibodies at different concentrations according to an embodiment of the present disclosure.

FIG. 4 is an SDS-PAGE identification result diagram of ROR2 expression in T47D cells and 231 cells according to an embodiment of the present disclosure.

FIG. 5 is a flow cytometry detection result diagram of different chimeric antibodies for detecting ROR2 in T47D cells according to an embodiment of the present disclosure.

FIG. 6 is a flow cytometry detection result diagram of different chimeric antibodies for detecting ROR2 in 231 cells according to an embodiment of the present disclosure.

FIG. 7 is a flow cytometry detection result diagram of using A12 chimeric antibody to detect the expression of ROR2 in hematological cancer patients according to an embodiment of the present disclosure.

FIG. 8 is a flow cytometry detection result diagram of using A12 chimeric antibody to detect the expression of ROR2 in lymphoma patients according to an embodiment of the present disclosure.

FIG. 9 is an immunoblotting detection result diagram of using different chimeric antibodies according to an embodiment of the present disclosure.

FIG. 10 is an immunoprecipitation detection result diagram of different chimeric antibodies for the detection of endogenous ROR2 protein according to an embodiment of the present disclosure.

FIG. 11 is an immunoprecipitation detection result diagram of different chimeric antibodies for the detection of exogenous ROR2 protein according to an embodiment of the present disclosure.

FIG. 12 is a structural diagram of an A12-car according to an embodiment of the present disclosure.

FIG. 13 is a flow cytometry detection result diagram of a ratio of lymphocytes capable of expressing CAR-T according to an embodiment of the present disclosure.

FIG. 14 is a killing result diagram of A12-Car-T on ROR2 expression-positive T47D cells and ROR2 expression-negative 231 cells according to an embodiment of the present disclosure.

### DETAILED DESCRIPTION OF THE EMBODIMENTS

The embodiments of the present disclosure are described in detail below, and examples of the embodiments are shown in the accompanying drawings. The same or similar reference numerals indicate the same or similar elements or elements with the same or similar functions. The embodiments described below with reference to the drawings are exemplary, and are intended to explain the present disclosure, and should not be understood as a limitation to the present disclosure.

During the process of describing the present disclosure, the relevant terms herein have been explained and described. These explanations and descriptions are only for the convenience of understanding the solution, and should not be regarded as a limitation to the protection solution of the present disclosure.

### Antibody

As used herein, the term “antibody” is an immunoglobulin molecule capable of binding to a specific antigen, including two light chains with a lighter molecular weight and two heavy chains with a heavier molecular weight. The heavy chain (H chain) and the light chain (L chain) are linked by disulfide bonds to form a tetrapeptide chain molecule. The amino acid sequence of the amino terminal (N-terminal) of the peptide chain varies greatly, which is called the variable region (V region), and the carboxyl terminal (C-terminal) is relatively stable with little change, called the constant region (C region). The V regions of the L chain and H chain are called VL and VH, respectively.

In the variable region, the amino acid composition and sequence of certain regions have a higher degree of change, which is called the hypervariable region (HVR). The hypervariable region is where the antigen and the antibody bind, so it is also called the complementarity-determining region (CDR). There are three CDR regions on both the heavy chain variable region and the light chain variable region.

The present disclosure uses the extracellular segment of ROR2 to obtain anti-ROR2 Fab antibody fragments with high specificity and high affinity through immunization. The antibody fragment can specifically bind to the ROR2 antigen, which can target the treatment of tumors and other diseases.

In some embodiments, the present disclosure provides an anti-ROR2 antibody or antigen-binding fragment, including a light chain variable region shown in CSASSSVSYMHWYQ, IYDTSKLAS and CQQWSSNPPTFGAG, and a heavy chain variable region shown in YTITSYLMHWV, LEWIGYINPYNDGTKYNEKFKDKAT and CARSDVYYGVRFAYWGQG. In some other embodiments, the anti-ROR2 antibody or antigen-binding fragment includes a light chain variable region shown in CKASQNVGTNVAWFQ, IYLASYRYS and CQQYNSYPLTFGGG, and a heavy chain variable region shown in YTFTNYWIQWM, LEWIGEINPSNGRTDYNEKFKNRAT and CANYRPGYWGQG. In some another embodiments, the anti-ROR2 antibody or antigen-binding fragment includes a light chain variable region shown in CSASSSISYMYWYQ, IYDTSILAS and CQQWSSYPFTFGSG, and a heavy chain variable region shown in YTFTSYLIHWV, LEWIGYINPYNDGTKYNEKFKDKAT and CARSDVYYGVRFAYWGQG. Take CSASSSVSYMHWYQ as an example. As the CDR region on the variable region of the light chain, it can bind to antigen with a high degree of specificity. The antibody or antigen-binding fragment having the above CDR region can specifically bind to the ROR2 antigen and perform the function of the ROR2 antibody.

In another embodiment, compared with any of the light chain variable regions and heavy chain variable regions described above, the antibody or antigen-binding fragment provided in the present disclosure has more than one conservative amino acid substitution. “Antigen-binding fragment” refers to an antibody fragment that retains the ability to specifically bind to an antigen (ROR2). “Conservative amino acid substitution” refers to the substitution of an amino acid with a residue that is biologically, chemically or structurally similar to another amino acid. Biologically similar means that the substitution does not destroy the biological activity of the ROR2 antibody or the ROR2 antigen. Structurally similar means that amino acids have side chains of similar length, such as alanine, glycine, or serine, or side chains of similar size. Chemically similar means that the amino acids have the same charge or are both hydrophilic or hydrophobic. For example, the hydrophobic residues isoleucine, valine, leucine or methionine are substituted for each other, alternatively, polar amino acids such as arginine for lysine, glutamic acid for aspartic acid, glutamine for asparagine, serine for threonine, and so on.

In some embodiments, the present disclosure provides an antibody or antigen-binding fragment including a light chain variable region sequence shown in SEQ ID NO: 1 and a heavy chain variable region sequence shown in SEQ ID NO: 2. In some other embodiments, compared with the amino acid sequence shown in SEQ ID NO: 1, the light chain variable region sequence of the antibody or antigen-binding fragment has more than one conservative amino acid substitution. In some embodiments, compared with the amino acid sequence shown in SEQ ID NO: 2, the heavy chain variable region sequence of the antibody or antigen-binding fragment has more than one conservative amino acid substitution.

In some embodiments, the present disclosure provides an antibody or antigen-binding fragment including a light chain variable region sequence shown in SEQ ID NO: 3 and a heavy chain variable region sequence shown in SEQ ID NO: 4. In some other embodiments, compared with the amino acid sequence shown in SEQ ID NO: 3, the light chain variable region sequence of the antibody or antigen-binding fragment has more than one conservative amino acid substitution. In some embodiments, compared with the amino acid sequence shown in SEQ ID NO: 4, the heavy chain variable region sequence of the antibody or antigen-binding fragment has more than one conservative amino acid substitution.

In some embodiments, the present disclosure provides an antibody or antigen-binding fragment including a light chain variable region sequence shown in SEQ ID NO: 5 and a heavy chain variable region sequence shown in SEQ ID NO: 6. In some other embodiments, compared with the amino acid sequence shown in SEQ ID NO: 5, the light chain variable region sequence of the antibody or antigen-binding fragment has more than one conservative amino acid substitution. In some embodiments, compared with the amino acid sequence shown in SEQ ID NO: 6, the heavy chain variable region sequence of the antibody or antigen-binding fragment has more than one conservative amino acid substitution.

These conservative amino acid substitutions will not change the biological function of the antibody or antigen-binding fragment. In some specific ways, these conservative amino acid substitutions can occur on amino acids other than the CDR regions in the heavy chain variable region and the light chain variable region.

The antibody including a light chain variable region sequence shown in SEQ ID NO: 1 and a heavy chain variable region sequence shown in SEQ ID NO: 2 is also named as A12 antibody. The light chain variable region sequence VL of the A12 antibody (SEQ ID NO: 1) is: DIVLTQSPAIMSASSPGEKVTMTCSASSSVSYMHWYQQKSGTSPKRWIYDTSKLASGVPA RFSGSGSGTSYSLTISSMEAEDAAIYYCQQWSSNPPTFGAGTKLELK. The heavy chain variable region sequence VH of the A12 antibody (SEQ ID NO: 2) is:

QVQLQQSGPELVKPGASVRMSCKAAGYTITSYLMTIWVKQRPGQDLEWIG
YINPYNDGTKYNEKFKDKATLTSDKSSSTAYMELSSLTSEDSAVYYCARS
DVYYGVRFAYWGQGTLVTVS.

The antibody including a light chain variable region sequence shown in SEQ ID NO: 3 and a heavy chain variable region sequence shown in SEQ ID NO: 4 is also named as B22 antibody. The light chain variable region sequence VL of the B22 antibody (SEQ ID NO: 3) is: DIVMTQSQKFMSTSVGDRVSVTCKASQNVGTNVAWFQQKPGQSPKPLIYLASYRYSGV PDRFTGSGSGTDFTLTISNVQSEDLAEYFCQQYNSYPLTFGGGTNLEIKR. The heavy chain variable region sequence VH of the B22 antibody (SEQ ID NO: 4) is:

QVQLQQPGAALVKPGASVKLSCKASGYTFTNYWIQWMKQRPGQGLEWIGE
INPSNGRTDYNEKFKNRATLTVDNSSTTAYMQLSSLTSEDSAVYYCANYR
PGYWGQGTSVTVSS.

The antibody including a light chain variable region sequence shown in SEQ ID NO: 5 and a heavy chain variable region sequence shown in SEQ ID NO: 6 is also named as B30 antibody. The light chain variable region sequence VL of the B30 antibody (SEQ ID NO: 5) is: DIVLTQSPVIMSASPGEKVTMTCSASSSISYMYWYQQKPGSSPRLLIYDTSILASGVPVR FSGSGSGTSYSLTISRMEAEDAATYYCQQWSSYPFTFGSGTKLEIK. The heavy chain variable region sequence VH of the B30 antibody (SEQ ID NO: 6) is:

QVQLQQSGPELVKPGASVRMSCKAAGYTFTSYLIHWVKQRPGQDLEWIGY
INPYNDGTKYNEKFKDKATLTSDKSSSTAYMELSSLTSEDSAVYYCARSD
VYYGVRFAYWGQGTLVTVSA.

In some embodiments, the present disclosure provides an anti-ROR2 antibody including a light chain shown in SEQ ID NO: 7 and a heavy chain shown in SEQ ID NO: 8.

SEQ ID NO: 7 is:

DIVLTQSPAIMSASPGEKVTMTCSASSSVSYMTIWYQQKSGTSPKRWIYD
TSKLASGVPARFSGSGSGTSYSLTISSMEAEDAAIYYCQQWSSNPPTFGA
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

SEQ ID NO: 8 is:

QVQLQQSGPELVKPGASVRMSCKAAGYTITSYLMHWVKQRPGQDLEWIGY
INPYNDGTKYNEKFKDKATLTSDKSSSTAYMELSSLTSEDSAVYYCARSD
VYYGVRFAYWGQGTLVTVSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK.

In some embodiments, the present disclosure provides an anti-ROR2 antibody including a light chain shown in SEQ ID NO: 9 and a heavy chain shown in SEQ ID NO: 10.

SEQ ID NO: 9 is:

DIVMTQSQKFMSTSVGDRVSVTCKASQNVGTNVAWFQQKPGQSPKPLIYL
ASYRYSGVPDRFTGSGSGTDFTLTISNVQSEDLAEYFCQQYNSYPLTFGG
GTNLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

SEQ ID NO: 10 is:

QVQLQQPGAALVKPGASVKLSCKASGYTFTNYWIQWMKQRPGQGLEWIGE
INPSNGRTDYNEKFKNRATLTVDNSSTTAYMQLSSLTSEDSAVYYCANYR
PGYWGQGTSVTVSSASTKGPSVFPLAPSSKSTSGGTAALGCLVKDYFPEP
VTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK.

In some embodiments, the present disclosure provides an anti-ROR2 antibody including a light chain shown in SEQ ID NO: 11 and a heavy chain shown in SEQ ID NO: 12.

SEQ ID NO: 11 is:

DIVLTQSPVIMSASPGEKVTMTCSASSSISYMYWYQQKPGSSPRLLIYDT
SILASGVPVRFSGSGSGTSYSLTISRMEAEDAATYYCQQWSSYPFTFGSG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

SEQ ID NO: 12 is:

QVQLQQSGPELVKPGASVRMSCKAAGYTFTSYLIHWVKQRPGQDLEWIGY
INPYNDGTKYNEKFKDKATLTSDKSSSTAYMELSSLTSEDSAVYYCARSD
VYYGVRFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K.

Polynucleotide, expression vector, recombinant cell

During the process of preparing or obtaining these antibodies, polynucleotides expressing these antibodies can be linked to different vectors and then expressed in different cells to obtain corresponding antibodies.

For this, the present disclosure also provides an isolated polynucleotide encoding the antibody or antigen-binding fragment described above.

In some embodiments, the isolated polynucleotides encode A12 antibody, B22 antibody, and B30 antibody, respectively.

The nucleotide sequence encoding the amino acid sequence shown in SEQ ID NO: 1 of the light chain variable region of the A12 antibody is (SEQ ID NO: 13):

gacatcgtgctgacccagtctccagccatcatgtctgctagccctggcga
gaaagtgacaatgacctgctccgcctcctcctccgtgtcctacatgcact
ggtatcagcagaagtccggcacctctcctaagcggtggatctacgacacc
tccaagctggctagcggagtgcctgccagattttccggctctggctctgg
cacctcttactccctgaccatctcctccatggaagccgaggatgccgcca
tctactactgccagcagtggtctagcaaccctcctacctttggcgctggc
accaagctggaactgaag.

The nucleotide sequence encoding the amino acid sequence shown in SEQ ID NO: 2 of the heavy chain variable region of the A12 antibody is (SEQ ID NO: 14):

caggttcagctgcagcagtctggacctgagctggttaagcctggtgcctc
cgtccggatgtcttgcaaggctgctggctacaccatcaccagctacctga
tgcactgggtcaagcagaggccaggccaggacttggagtggatcggctac
atcaacccctacaacgacggcaccaagtacaacgagaagttcaaggacaa
ggctaccctgacctccgacaagtcctcctccaccgcctacatggaactgt
ccagcctgacctctgaggactccgccgtgtactactgcgccagatccgat
gtgtactatggcgtcagattcgcctactggggccagggcacactggtcac
agtttct.

The nucleotide sequence encoding the amino acid sequence shown in SEQ ID NO: 3 of the light chain variable region of the B22 antibody is (SEQ ID NO: 15):

gacatcgtgctgacccagtctccagccatcatgtctgctagccctggcga
gaaagtgacaatgacctgctccgcctcctcctccgtgtcctacatgcact
ggtatcagcagaagtccggcacctctcctaagcggtggatctacgacacc
tccaagctggctagcggagtgcctgccagattttccggctctggctctgg
cacctcttactccctgaccatctcctccatggaagccgaggatgccgcca
tctactactgccagcagtggtctagcaaccctcctacctttggcgctggc
accaagctggaactgaag.

The nucleotide sequence encoding the amino acid sequence shown in SEQ ID NO: 4 of the heavy chain variable region of the B22 antibody is (SEQ ID NO: 16):

caggttcagttgcagcaacctggcgctgctctggttaagcctggcgcttc
tgtgaagctgtcctgcaaggcttccggctacaccttcaccaactattgga
tccagtggatgaagcagcggccaggccaaggcctggaatggatcggagag
atcaacccttctaacggccggaccgactacaacgagaagttcaagaaccg
ggctaccctgaccgtggacaactcttctaccaccgcctacatgcagctgt
ccagcctgacctctgaggactccgccgtgtactactgcgccaactacaga
cctggctattggggccagggcacctctgtgacagtctcttct.

The nucleotide sequence encoding the amino acid sequence shown in SEQ ID NO: 5 of the light chain variable region of the B30 antibody is (SEQ ID NO: 17):

gacatcgtgatgacccagagccagaaattcatgtccacctccgtgggcga
cagagtgtccgtgacatgcaaggcctctcagaacgtgggcaccaacgtgg
cctggttccagcagaaacctggccagtctcctaagcctctgatctacctg
gcctcctaccggtactctggcgtgcccgatagattcaccggctctggatc
tggcaccgacttcaccctgaccatctccaacgtgcagtctgaggacctgg
ccgagtacttctgccagcagtacaacagctaccctctgacctttggcgga
ggcaccaacctggaaatcaag.

The nucleotide sequence encoding the amino acid sequence shown in SEQ ID NO: 6 of the heavy chain variable region of the B30 antibody is (SEQ ID NO: 18):

caggttcagctgcagcagtctggacctgagctggttaagcctggtgcctc
cgtccggatgtcttgcaaggctgccggctacaccttcaccagctacctga
tccactgggtcaagcagaggccaggccaggacttggagtggatcggctac
atcaacccctacaacgacggcaccaagtacaacgagaagttcaaggacaa
ggctaccctgacctccgacaagtcctcctccaccgcctacatggaactgt
ccagcctgacctctgaggactccgccgtgtactactgcgccagatccgat
gtgtactatggcgtcagattcgcctactggggccagggcacactggtcac
agtttctgct.

The nucleotide sequence encoding the amino acid sequence shown in SEQ ID NO: 7 of the light chain of the A12 antibody is (SEQ ID NO: 19):

gacatcgtgctgacccagtctccagccatcatgtctgctagccctggcga
gaaagtgacaatgacctgctccgcctcctcctccgtgtcctacatgcact
ggtatcagcagaagtccggcacctctcctaagcggtggatctacgacacc
tccaagctggctagcggagtgcctgccagattttccggctctggctctgg
cacctcttactccctgaccatctcctccatggaagccgaggatgccgcca
tctactactgccagcagtggtctagcaaccctcctacctttggcgctggc
accaagctggaactgaagagaacagtggccgctcctagcgtgttcatctt
cccaccttccgacgagcagctgaagtctggcacagcctctgtcgtgtgcc
tgctgaacaacttctaccccagagaagccaaggtgcagtggaaggtggac
aacgccctgcagagcggcaatagccaagagagcgtgaccgagcaggacag
caaggactctacctacagcctgagcagcaccctgacactgagcaaggccg
actacgagaagcacaaagtgtacgcctgcgaagtgacccaccagggcctt
tctagccctgtgaccaagagcttcaaccggggcgaatgt.

The nucleotide sequence encoding the amino acid sequence shown in SEQ ID NO: 8 of the heavy chain of the A12 antibody is (SEQ ID NO: 20):

caggttcagctgcagcagtctggacctgagctggttaagcctggtgcctc
cgtccggatgtcttgcaaggctgctggctacaccatcaccagctacctga
tgcactgggtcaagcagaggccaggccaggacttggagtggatcggctac
atcaacccctacaacgacggcaccaagtacaacgagaagttcaaggacaa
ggctaccctgacctccgacaagtcctcctccaccgcctacatggaactgt
ccagcctgacctctgaggactccgccgtgtactactgcgccagatccgat
gtgtactatggcgtcagattcgcctactggggccagggcacactggtcac
agtttctgcctctacaaagggccctagtgtgttccctctggctcccagca
gcaagtctacatctggcggaacagccgctctgggctgcctggtcaaggat
tactttcccgagcctgtgaccgtgtcctggaatagcggagcactgacaag
cggcgtgcacacctttccagctgtgctgcaaagcagcggcctgtactctc
tgagcagcgtggtcacagtgcctagctctagcctgggcacccagacctac
atctgcaatgtgaaccacaagcctagcaacaccaaggtggacaagaaggt
ggaacccaagagctgcgacaagacccacacctgtcctccatgtcctgctc
cagaactgctcggcggaccttccgtgttcctgtttcctccaaagcctaag
gacaccctgatgatcagcagaacccctgaagtgacctgcgtggtggtgga
tgtgtcccacgaggatcccgaagtgaagttcaattggtacgtggacggcg
tggaagtgcacaacgccaagaccaagcctagagaggaacagtacaacagc
acctacagagtggtgtccgtgctgaccgtgctgcaccaggattggctgaa
cggcaaagagtacaagtgcaaggtgtccaacaaggccctgcctgctccta
tcgagaaaaccatcagcaaggccaagggccagcctagggaaccccaggtt
tacacactgcctccaagcagggacgagctgaccaagaatcaggtgtccct
gacctgcctcgtgaagggcttctacccttccgatatcgccgtggaatggg
agagcaatggccagcctgagaacaactacaagacaacccctcctgtgctg
gacagcgacggctcattcttcctgtacagcaagctgacagtggacaagtc
cagatggcagcagggcaacgtgttcagctgcagcgtgatgcacgaggccc
tgcacaaccactacacccagaagtccctgagcctgtctcctggcaaa.

The nucleotide sequence encoding the amino acid sequence shown in SEQ ID NO: 9 of the light chain of the B22 antibody is (SEQ ID NO: 21):

gacatcgtgatgacccagagccagaaattcatgtccacctccgtgggcga
cagagtgtccgtgacatgcaaggcctctcagaacgtgggcaccaacgtgg
cctggttccagcagaaacctggccagtctcctaagcctctgatctacctg
gcctcctaccggtactctggcgtgcccgatagattcaccggctctggatc
tggcaccgacttcaccctgaccatctccaacgtgcagtctgaggacctgg
ccgagtacttctgccagcagtacaacagctaccctctgacctttggcgga
ggcaccaacctggaaatcaagagaacagtggccgctcctagcgtgttcat
cttcccaccttccgacgagcagctgaagtctggcacagcctctgtcgtgt
gcctgctgaacaacttctaccccagagaagccaaggtgcagtggaaggtg
gacaacgccctgcagagcggcaatagccaagagagcgtgaccgagcagga
cagcaaggactctacctacagcctgagcagcaccctgacactgagcaagg
ccgactacgagaagcacaaagtgtacgcctgcgaagtgacccaccagggc
ctttctagccctgtgaccaagagcttcaaccggggcgaatgt.

The nucleotide sequence encoding the amino acid sequence shown in SEQ ID NO: 10 of the heavy chain of the B22 antibody is (SEQ ID NO: 22):

caggttcagttgcagcaacctggcgctgctctggttaagcctggcgcttc
tgtgaagctgtcctgcaaggcttccggctacaccttcaccaactattgga
tccagtggatgaagcagcggccaggccaaggcctggaatggatcggagag
atcaacccttctaacggccggaccgactacaacgagaagttcaagaaccg
ggctaccctgaccgtggacaactcttctaccaccgcctacatgcagctgt
ccagcctgacctctgaggactccgccgtgtactactgcgccaactacaga
cctggctattggggccagggcacctctgtgacagtctcttctgcctctac
aaagggccctagtgtgttccctctggctcccagcagcaagtctacatctg
gcggaacagccgctctgggctgcctggtcaaggattactttcccgagcct
gtgaccgtgtcctggaatagcggagcactgacaagcggcgtgcacacctt
tccagctgtgctgcaaagcagcggcctgtactctctgagcagcgtggtca
cagtgcctagctctagcctgggcacccagacctacatctgcaatgtgaac
cacaagcctagcaacaccaaggtggacaagaaggtggaacccaagagctg
cgacaagacccacacctgtcctccatgtcctgctccagaactgctcggcg
gaccttccgtgttcctgtttcctccaaagcctaaggacaccctgatgatc
agcagaacccctgaagtgacctgcgtggtggtggatgtgtcccacgagga
tcccgaagtgaagttcaattggtacgtggacggcgtggaagtgcacaacg
ccaagaccaagcctagagaggaacagtacaacagcacctacagagtggtg
tccgtgctgaccgtgctgcaccaggattggctgaacggcaaagagtacaa
gtgcaaggtgtccaacaaggccctgcctgctcctatcgagaaaaccatca
gcaaggccaagggccagcctagggaaccccaggtttacacactgcctcca
agcagggacgagctgaccaagaatcaggtgtccctgacctgcctcgtgaa
gggcttctacccttccgatatcgccgtggaatgggagagcaatggccagc
ctgagaacaactacaagacaacccctcctgtgctggacagcgacggctca
ttcttcctgtacagcaagctgacagtggacaagtccagatggcagcaggg
caacgtgttcagctgcagcgtgatgcacgaggccctgcacaaccactaca
cccagaagtccctgagcctgtctcctggcaaa.

The nucleotide sequence encoding the amino acid sequence shown in SEQ ID NO: 11 of the light chain of the B30 antibody is (SEQ ID NO: 23):

gacatcgtgctgacccagtctccagtgatcatgtccgcttctcccggcga
gaaagtgacaatgacctgctccgcctcctccagcatctcctacatgtact
ggtatcagcagaagcccggctcctctcctcggctgctgatctacgatacc
tccatcctggcttccggcgtgccagtgcggttttctggttctggctctgg
cacctcctacagcctgaccatctccagaatggaagccgaggacgccgcca
cctactactgtcagcagtggtctagctaccccttcacctttggctccggc
accaagctggaaatcaagagaacagtggccgctcctagcgtgttcatctt
cccaccttccgacgagcagctgaagtctggcacagcctctgtcgtgtgcc
tgctgaacaacttctaccccagagaagccaaggtgcagtggaaggtggac
aacgccctgcagagcggcaatagccaagagagcgtgaccgagcaggacag
caaggactctacctacagcctgagcagcaccctgacactgagcaaggccg
actacgagaagcacaaagtgtacgcctgcgaagtgacccaccagggcctt
tctagccctgtgaccaagagcttcaaccggggcgaatgt.

The nucleotide sequence encoding the amino acid sequence shown in SEQ ID NO: 12 of the heavy chain of the B30 antibody is (SEQ ID NO: 24):

caggttcagctgcagcagtctggacctgagctggttaagcctggtgcctc
cgtccggatgtcttgcaaggctgccggctacaccttcaccagctacctga
tccactgggtcaagcagaggccaggccaggacttggagtggatcggctac
atcaacccctacaacgacggcaccaagtacaacgagaagttcaaggacaa
ggctaccctgacctccgacaagtcctcctccaccgcctacatggaactgt
ccagcctgacctctgaggactccgccgtgtactactgcgccagatccgat
gtgtactatggcgtcagattcgcctactggggccagggcacactggtcac
agtttctgctgcctctacaaagggccctagtgtgttccctctggctccca
gcagcaagtctacatctggcggaacagccgctctgggctgcctggtcaag
gattactttcccgagcctgtgaccgtgtcctggaatagcggagcactgac
aagcggcgtgcacacctttccagctgtgctgcaaagcagcggcctgtact
ctctgagcagcgtggtcacagtgcctagctctagcctgggcacccagacc
tacatctgcaatgtgaaccacaagcctagcaacaccaaggtggacaagaa
ggtggaacccaagagctgcgacaagacccacacctgtcctccatgtcctg
ctccagaactgctcggcggaccttccgtgttcctgtttcctccaaagcct
aaggacaccctgatgatcagcagaacccctgaagtgacctgcgtggtggt
ggatgtgtcccacgaggatcccgaagtgaagttcaattggtacgtggacg
gcgtggaagtgcacaacgccaagaccaagcctagagaggaacagtacaac
agcacctacagagtggtgtccgtgctgaccgtgctgcaccaggattggct
gaacggcaaagagtacaagtgcaaggtgtccaacaaggccctgcctgctc
ctatcgagaaaaccatcagcaaggccaagggccagcctagggaaccccag
gtttacacactgcctccaagcagggacgagctgaccaagaatcaggtgtc
cctgacctgcctcgtgaagggcttctacccttccgatatcgccgtggaat
gggagagcaatggccagcctgagaacaactacaagacaacccctcctgtg
ctggacagcgacggctcattcttcctgtacagcaagctgacagtggacaa
gtccagatggcagcagggcaacgtgttcagctgcagcgtgatgcacgagg
ccctgcacaaccactacacccagaagtccctgagcctgtctcctggcaa
a.

In some embodiments, the isolated polynucleotide has at least 90% or more homology with the aforementioned amino acid encoding sequence, preferably more than 95% homology, and more preferably 98%, 99% or more homology.

The present disclosure also provides an expression vector, including the isolated polynucleotide described above. When the isolated polynucleotide is linked to the vector, the polynucleotide can be directly or indirectly linked to the control elements on the vector, as long as these control elements can control the translation and expression of the polynucleotide. These control elements can come directly from the vector itself, or can be exogenous, that is, they do not come from the vector itself. These control elements may be promoters, enhancers, terminators, etc., as long as they can regulate gene expression. The polynucleotide only needs to be operably linked to the control elements. “Operably linked” herein refers to linking an exogenous gene to a vector, such that the control elements in the vector, such as transcription control sequences and translation control sequences, etc., can perform their expected functions of regulating the transcription and translation of foreign genes. The polynucleotides encoding the heavy chain and light chain of the antibody can be inserted into different vectors independently, and it is common to insert into the same vector. Commonly used vectors can be, for example, plasmids, bacteriophages, etc., such as pcDNA plasmids.

The present disclosure also provides a recombinant cell including the expression vector. The expression vector can be introduced into mammalian cells (such as 293F cells, CHO cells, etc.) to construct the recombinant cells, and then uses these recombinant cells to express the antibody or antigen-binding fragment provided in the present disclosure. By culturing the recombinant cells, the corresponding antibodies can be obtained.

Pharmaceutical composition, kit, pharmaceutical use and use in the preparation of kit

The present disclosure also provides a pharmaceutical composition including the antibody or antigen-binding fragment described above and a pharmaceutically acceptable carrier.

The anti-ROR2 antibody provided herein can be incorporated into the pharmaceutical composition suitable for administration to a subject. Generally, the pharmaceutical composition includes the anti-ROR2 antibody provided herein and a pharmaceutically acceptable carrier. The “pharmaceutically acceptable carrier” can include any and all solvents, dispersion media, coatings, antibacterial and antifungal agents, isotonic and absorption delaying agents, and the like that are physiologically compatible. Specific examples may be one or more of water, saline, phosphate buffered saline, glucose, glycerol, ethanol, etc., and combinations thereof. In many cases, isotonic agents are included in the pharmaceutical composition, such as sugars, polyols (such as mannitol, sorbitol), or sodium chloride. The pharmaceutically acceptable carrier can also include minor amounts of auxiliary substances, such as wetting or emulsifying agents, preservatives or buffers, to extend the shelf life or efficacy of the antibody.

For example, the antibody of the present disclosure can be incorporated into the pharmaceutical composition suitable for parenteral administration (e.g., intravenous, subcutaneous, intraperitoneal, intramuscular). The pharmaceutical composition can be prepared into various forms, such as liquid, semi-solid and solid dosage forms, or the like, including but not limited to liquid solution (for example, injection solution and infusion solution), dispersion or suspension, tablet, pill, powder, liposome, and suppository. Typical pharmaceutical composition is in the form of injection solution or infusion solution. The antibody can be administered by intravenous infusion or injection, or intramuscular or subcutaneous injection.

The anti-ROR2 antibody herein can also be made into a part of a kit or other diagnostic reagents as needed. According to an embodiment of the present disclosure, the present disclosure also provides a kit including the ROR2 antibody described above. The kit provided in the present disclosure, for example, can be used for immunoblotting, immunoprecipitation, etc., involving the use of ROR2 antigen and antibody specific binding properties. The kit can include any one or more of the following: antagonist, anti-ROR2 antibody or drug reference material; protein purification column; immunoglobulin affinity purification buffer; cell assay diluent; instruction or literature, or the like. The anti-ROR2 antibody can be used in different types of diagnostic tests, for example, can detect the presence of various diseases or drugs, toxins or other proteins in vitro or in vivo. For example, the subject's serum or blood can be tested to test related diseases. Such related diseases can include ROR2 related diseases, such as various cancers and so on. The antibody provided herein can also be used for radioimmunoassay and radioimmunotherapy of cancer.

These cancers or tumors can be any unregulated cell growth, specifically, lung cancer, gastric cancer, pancreatic cancer, ovarian cancer, liver cancer, breast cancer, colorectal cancer, lymphoma, blood cancer, and so on.

When using the anti-ROR2 antibody provided in the present disclosure to treat cancer, the anti-ROR2 antibody provided in the present disclosure can be provided to the subject. To this end, the present disclosure provides a method for treating cancer, including administering the antibody or antigen-binding fragment thereof provided in the present disclosure to a subject in need.

The solution of the present disclosure will be explained below in conjunction with embodiments. Those skilled in the art will understand that the following embodiments are only used to illustrate the present disclosure, and should not be regarded as limiting the scope of the present disclosure. Where specific techniques or conditions are not indicated in the examples, the procedures shall be carried out in accordance with the techniques or conditions described in the literature in the field or in accordance with the product specification. The reagents or instruments used without the manufacturer's indication are all conventional products that can be purchased commercially.

Through research, the present disclosure has developed ROR2-based antibody-based immunotherapy methods against multiple tumor cells (even tumor stem cells), including ROR2 monoclonal antibody, and a novel ROR2-Car T cell therapy technology.

### Embodiment 1

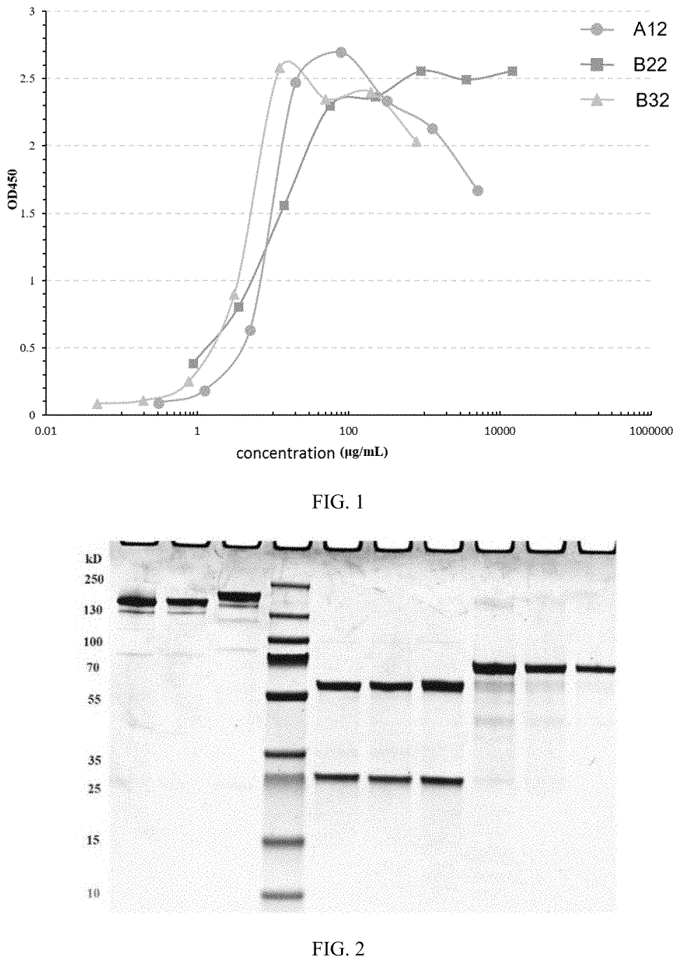

We use ROR2 protein, immunize mice and use phage library technology to screen and obtain B30, B22 and A12 antibodies. The ELISA detection results of these antibodies are shown in FIG. 1. In FIG. 1, the abscissa represents the concentration of different antibodies, and the ordinate represents the measured OD450 value.

The amino acid sequencing is performed on these antibody samples. The amino acid sequence of the light chain variable region of the antibody and the amino acid sequence of the heavy chain variable region of the antibody are SEQ ID NO: 1 and SEQ ID NO: 2, respectively, and the antibody is named as A12 antibody. The amino acid sequence of the light chain variable region of the antibody and the amino acid sequence of the heavy chain variable region of the antibody are SEQ ID NO: 3 and SEQ ID NO: 4, respectively, and the antibody is named as B22 antibody. The amino acid sequence of the light chain variable region of the antibody and the amino acid sequence of the heavy chain variable region of the antibody are SEQ ID NO: 5 and SEQ ID NO: 6, respectively, and the antibody is named as B30 antibody.

It was detected that the amino acid sequence of the light chain of the A12 antibody is SEQ ID NO: 7, and the amino acid sequence of the heavy chain of the A12 antibody is SEQ ID NO: 8. The amino acid sequence of the light chain of the B22 antibody is SEQ ID NO: 9, and the amino acid sequence of the heavy chain of the B22 antibody is SEQ ID NO: 10. The amino acid sequence of the light chain of the B30 antibody is SEQ ID NO: 11, and the amino acid sequence of the heavy chain of the B30 antibody is SEQ ID NO: 12.

### Embodiment 2

(1) Expression and Purification of ROR2 Chimeric Antibody

The amino acid sequences of the variable regions of the A12 antibody, the B22 antibody and the B30 antibody were used to deduce their DNA sequences, and the expression sequence of the chimeric antibody was established after species optimization according to the expression vector, and cloned into the pcDNA3.4 vector.

Only codon optimization is performed on the amino acid sequence of the antibody that is not changed after optimization, and the optimized nucleotide sequence encoding the amino acid sequence is easier to be expressed in mammalian cells. Therefore, for example, when the A12 chimeric antibody is expressed, its amino acid sequence is the same as the above-mentioned A12 antibody amino acid sequence, with the difference that the nucleotide sequence encoding the A12 chimeric antibody is optimized.

Specifically, the nucleotide sequence encoding the light chain of the A12 chimeric antibody is SEQ ID NO: 19, and the nucleotide sequence encoding the heavy chain of the A12 chimeric antibody is SEQ ID NO: 20.

The nucleotide sequence encoding the light chain of the B22 chimeric antibody is SEQ ID NO: 21, and the nucleotide sequence encoding the heavy chain of the B22 chimeric antibody is SEQ ID NO: 22.

The nucleotide sequence encoding the light chain of the B30 chimeric antibody is SEQ ID NO: 23, and the nucleotide sequence encoding the heavy chain of the B22 chimeric antibody is SEQ ID NO: 24.

Then the constructed plasmid was transfected into 293F cells with PEI and expressed for 48 hours. After 48 hours, the supernatant was recovered, and purified and chromatographed/concentrated on a Protein A column. The purified protein was identified by SDS-PAGE gel, and the result is shown in FIG. 2. FIG. 2 shows the SDS-PAGE identification results of ROR2-B30 (SA120-1), B22 (SA120-2), and A12 (SA120-3) purified antibodies. The results show that the purified antibodies have high purity.

In FIG. 1, from left to right, the first, second, and third columns represent the SDS-PAGE gel identification results of non-reduced A12, B22, and B30 antibody samples with a loading amount of 2.0 μg. The fourth column represents protein marker. The fifth, sixth, and seventh columns represent the SDS-PAGE gel identification results of reduced A12, B22, and B30 antibody samples with a loading amount of 2.0 μg. The eighth, ninth, and tenth columns represent the SDS-PAGE gel results of reduced fetal bovine serum albumin (BSA) samples with loading amounts of 0.5 μg, 1.0 μg, and 2 μg respectively.

It can be seen from FIG. 2 that the reduced and non-reduced A12, B22, and B30 chimeric antibody bands displayed in the SDS-PAGE gel are the same. The gray value of the band is close to 2 μg of reduced fetal bovine serum albumin (BSA) sample, which shows that the purified antibody has high purity.

(2) ELASA Verifies the Affinity of the Purified ROR2 Chimeric Antibody

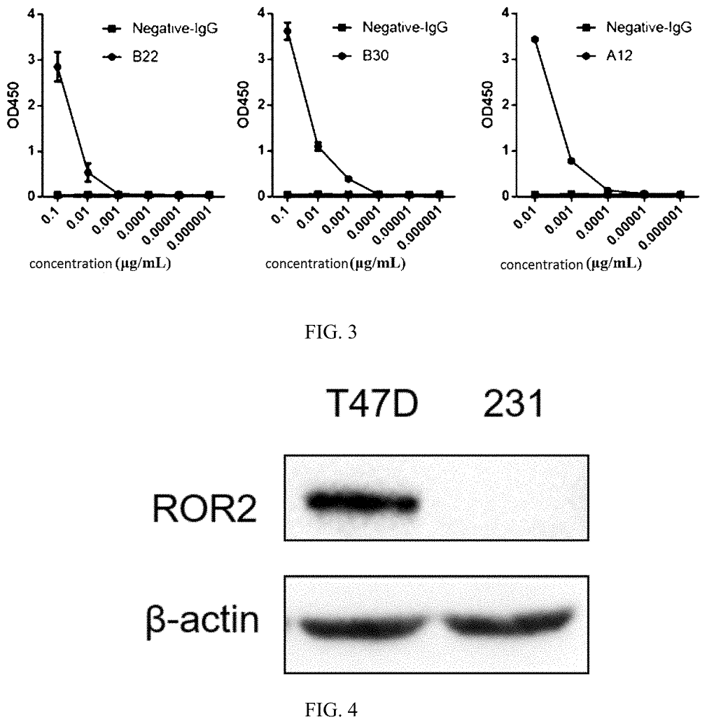

The 96-well plate was coated with ROR2 extracellular protein overnight. After blocking with 5% milk, the purified antibody was diluted to different concentrations and added to different wells for ELASA detection. OD450 is shown in FIG. 3. FIG. 3 shows the OD450 values of different concentrations of B30 chimeric antibody, B22 chimeric antibody and A12 chimeric antibody. It can be seen that when the concentration of chimeric antibody is low (0.001 μg/ml or 0.0001 μg/ml), the antibody already has affinity, which also shows that the chimeric antibody can be used for ELISA and has a higher affinity.

(3) Flow Cytometry Detects the Binding Capacity of Antibody

The purified antibody was subjected to flow cytometry to detect T47D cells (positive ROR2 expression, FIG. 4) and 231 breast cancer cells (negative ROR2 expression, FIG. 4). The results are shown in FIG. 4 to FIG. 6. The flow cytometry results showed that the three chimeric antibodies could specifically bind to the ROR2 membrane protein.

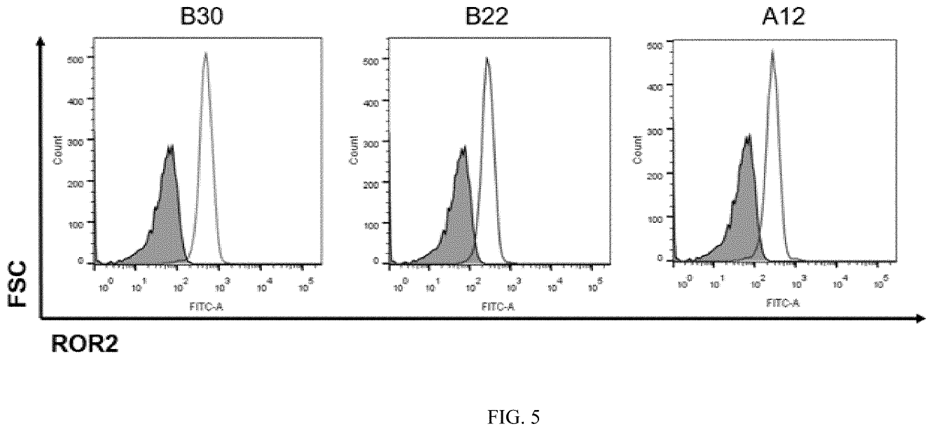

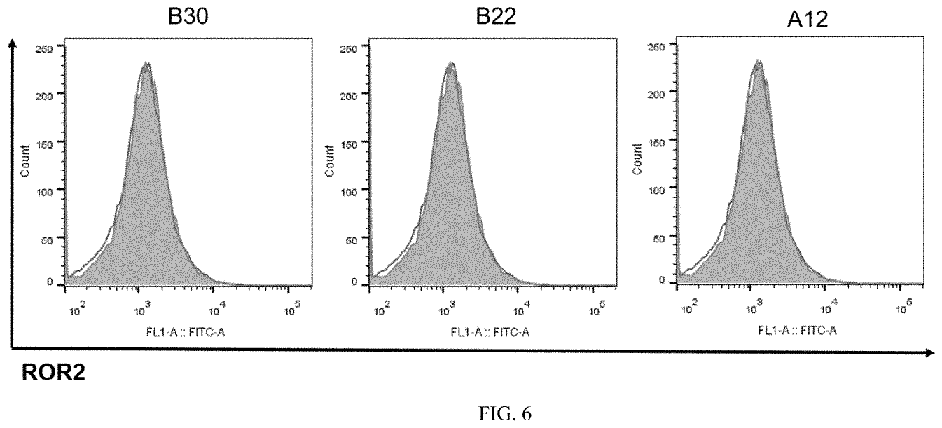

FIG. 4 shows the detection of ROR2 expression levels in T47D cells and 231 cells with ROR2 antibody of the company Santa Cruz. FIG. 5 shows the results of the flow cytometric detection of the ROR2 expression level of T47D with three chimeric antibodies, and the concentrations of the three chimeric antibodies are all 10 μg/ml. FIG. 6 shows the results of the flow cytometric detection of the ROR2 expression level of 231 with three chimeric antibodies, and the concentrations of the three chimeric antibodies are all 10 μg/ml.

### Embodiment 3

(1) A12 Chimeric Antibody Detects ROR2 Levels of Cancer Cells in Different Blood Cancer Patients and Lymphomas

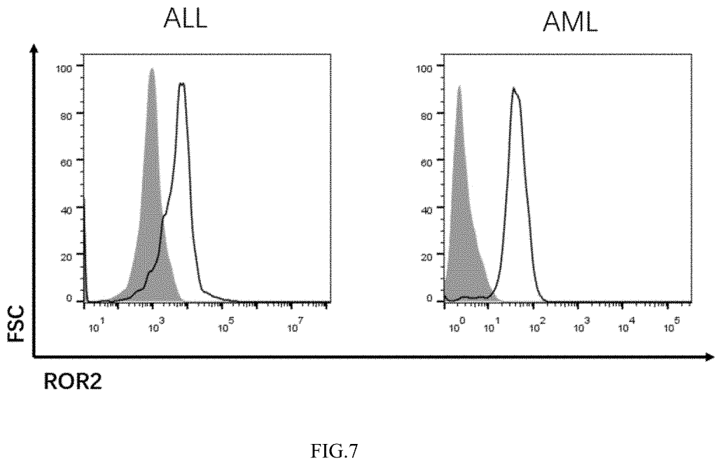

The peripheral blood of patients with different blood cancers were obtained, Ficoll was used to separate the lymphocytes in the blood, and then the lymphocytes were added to the A12 chimeric antibody (concentration of 3 μg/ml), incubated for 1 hour, then Alexa-488-conjugated anti-mouse IgG secondary antibody was incubated and flow cytometry was performed. The results are shown in FIG. 7. In FIG. 7, the results corresponding to ALL represent the flow cytometry results of samples from patients with acute lymphoblastic leukemia, and the results corresponding to AML, represent the results of flow cytometry from samples from patients with acute myeloid leukemia.

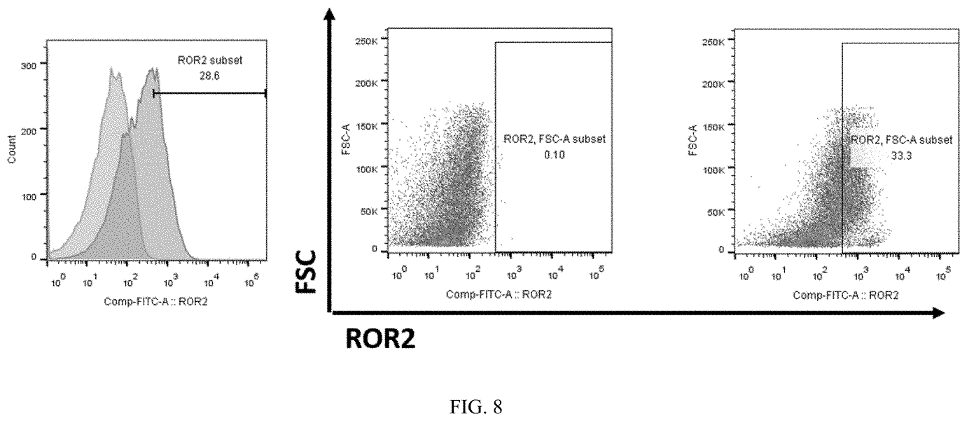

The tissues of lymphoma patients were obtained from the hospital, and then the patiend-derived-xenograft (PDX) model was established. The tumor tissues grown from PDX mice were taken and the tissues were separated into single cells with the human dissociation kit. The single cells were added to the A12 chimeric antibody (concentration of 3 μg/ml), incubated for 1 hour, then Alexa-488-conjugated anti-mouse IgG secondary antibody was incubated, and flow cytometry was performed to check the ROR2 level of tumor cells. The results are shown in FIG. 8. The left image of FIG. 8 shows the peak graph of ROR2 fluorescence intensity of tumor cells in lymphoma patients, and the two scatter plots on the right show the distribution of cell fluorescence intensity of tumor cells in blank tubes and B16 antibody stained tumor cells in lymphoma patients.

(2) ROR2 Chimeric Antibody can be Applied to Western Blot (WB)

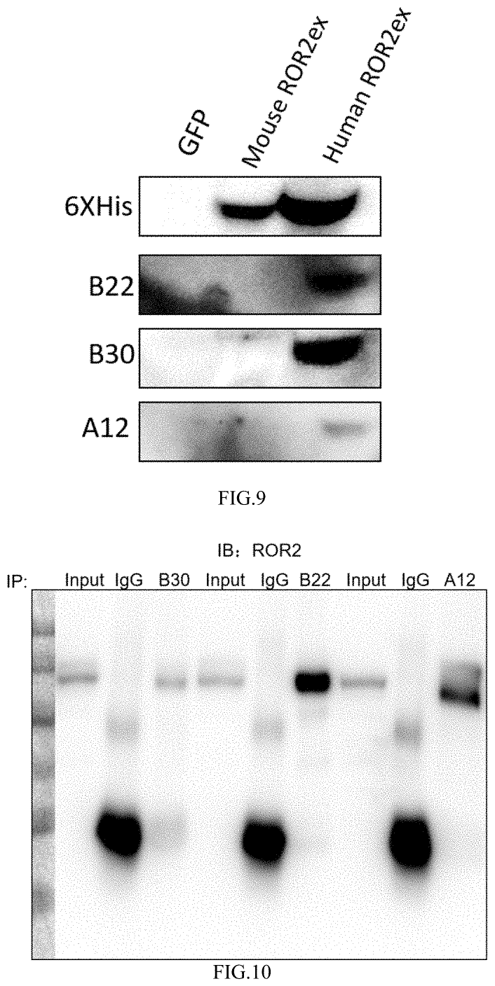

The mouse-derived ROR2 extracellular end (mouse ROR2ex) and human ROR2 extracellular end (human ROR2ex) with His-tagged plasmids and empty GFP were transfected into ROR2 knockout 293T cells (293T-ROR2-KO). After 48 hours, the cells were lysed and the total protein was extracted. After denaturation, the protein was separated by SDS-PAGE gel and subjected to immunoblotting reaction. Three ROR2 chimeric antibodies (5 μg/ml) were used to detect the expression of ROR2. The results are shown in FIG. 9, indicating that the ROR2-B30 chimeric antibody, B22 chimeric antibody and A12 chimeric antibody can all be applied to western blot detection, and can only recognize the extracellular end of the human ROR2 protein.

(3) ROR2 Chimeric Antibody can be Applied to Immunoprecipitation (IP)

In order to determine whether the ROR2 chimeric antibody can be applied to immunoprecipitation (IP), 293T cells with positive ROR2 expression were used for IP. After the cells were lysed, IP was performed with B30 chimeric antibody, B22 chimeric antibody and A12 chimeric antibody, respectively. The protein after IP was detected by immunoblotting with Santa Cruz's ROR2 (Cat. No.: sc-374174) antibody. IgG antibody is used as a negative control antibody (ROR2 cannot be precipitated), and Input is a cell lysate sample that has not been immunoprecipitated (ROR2 protein can be expressed). The results are shown in FIG. 10, indicating that all three antibodies can be applied to IP and can detect endogenous ROR2.

To further verify whether the ROR2 antibody can perform IP, 293T cells were transfected with Flag-tagged pcDNA-ROR2-Flag plasmid. The Flag tag is a polypeptide fragment composed of eight hydrophilic amino acids DYKDDDDK, which can be fused with the protein without occupying the protein epitope or structural domain, avoiding affecting the function, secretion, and transportation of the protein. The Flag tag was used to mark the target protein. If the tag is expressed, the target protein will also be expressed. Then B30 chimeric antibody, B22 chimeric antibody, A12 chimeric antibody and Flag were used for IP, respectively the Flag and B30 chimeric antibody, B22 chimeric antibody, and A12 chimeric antibody were used for immunoblotting (IB). The results are shown in FIG. 11, indicating that regardless of whether it was ROR2 antibody or Flag-labeled antibody for IP, ROR2 (130 kD) bands could be detected in the IB results.

It can be seen from the above experiments that by using the spleens of immunized mice to construct a phage library, screening high-affinity monoclonal antibody that specifically recognizes ROR2. Then, the diagnostic function test was performed to verify that the antibody that we screened to specifically recognize ROR2 can be used as a help for the clinically accurate diagnosis of tumor cells, which will lay the foundation for future clinical treatment of ROR2-positive tumor patients, and can also determine the signal pathways affected by the ROR2 antibody, and provide biomarkers for predicting the patient's response to drugs in future clinical trials.

### Embodiment 4

The experiment further verified the ROR2 antibody provided in the present disclosure and developed ROR2-chimeric antigen receptor (CAR) T cell therapy technology for targeted therapy of diseases.

### 1. Construction of ROR2-A12 CAR

The second-generation CAR sequence was used to construct ROR2-CAR with the high-affinity anti-ROR2 antibody A12.

### 2. A12 CAR Structure

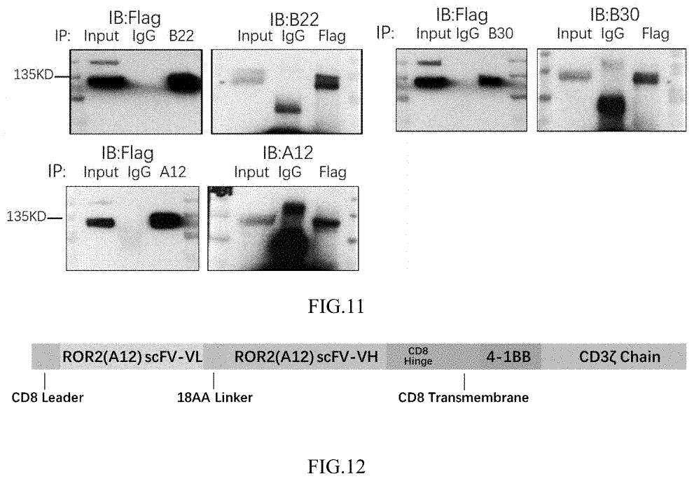

A12 CAR is composed of CD8 leader, A12 scFv-VL, 18 amino acid linker, A12 scFv-VH, CD8 hinge region, CD8 transmembrane region, 4-1BB costimulatory factor structure, and CD3ζ chain structure, as shown in FIG. 12.

### 3. CAR-T can Bind to ROR2

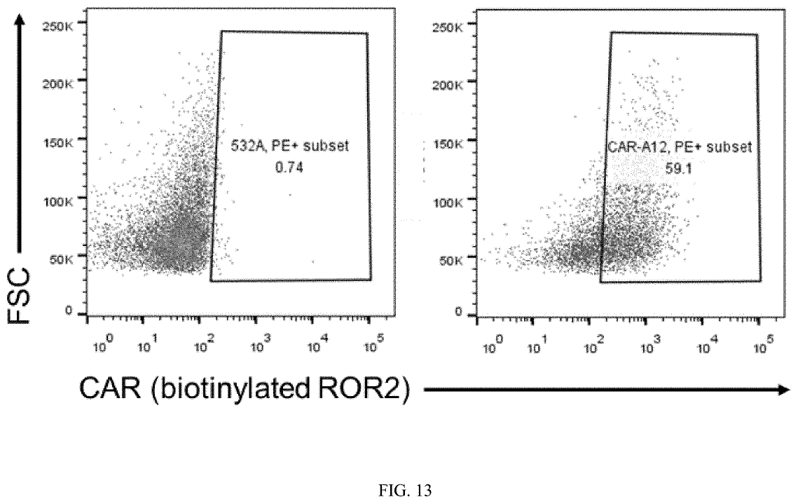

The A12-CAR-T cells were incubated with biotinylated ROR2 protein at a final concentration of 2 μg/ml for 30 minutes on ice. Then, the ROR2 protein was labeled with affinity streptomycin with PE fluorescence and placed on ice for 15 minutes. Flow cytometry was used to detect the ratio of CAR-T that can bind to the ROR2 protein. The flow cytometry results are shown in FIG. 13. In FIG. 13, the left image is a scatter plot of a blank tube, and the right image is a scatter plot of ROR2 protein binding antibody staining, indicating that 59.1% of lymphocytes express A12-CAR T.

### 4. CAR-T Kills Target Cells In Vitro

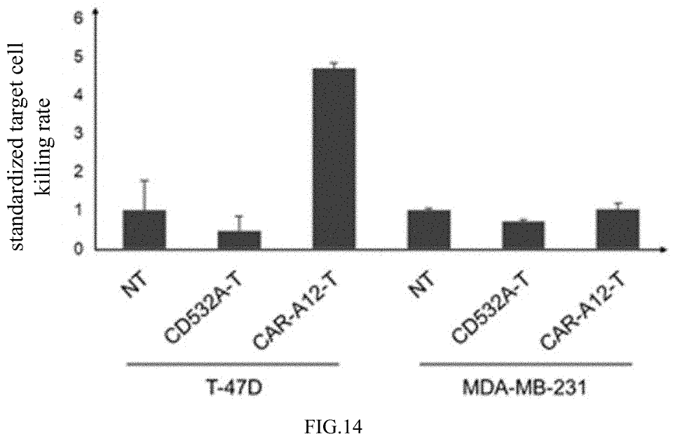

Untransfected T cells (NT), transfected empty-CD532A-vector T, or transfected A12-CAR-vector T cells were co-cultured with two different target cells in 96-well plates, respectively. One of the target cells is the T-47D breast cancer cell line that expresses ROR2, and the other of the target cells is the MDA-MB-231 breast cancer cell line that does not express ROR2. 12,000 target cells and 120,000 effect T cells per well, effect (E): target (T)=10: 1, were cultured for 12 hours, and then the lactate dehydrogenase release test was used to detect the killing of the target cells. The killing rate is calculated according to the formula % cytotoxicity=(experimental group-spontaneous effector cell-spontaneous target cell)/(maximum target cell-spontaneous target cell)×100. The resulting kill rate is then standardized on the basis of the kill rate of the target cells in the untransfected T cell (NT) group. The results are shown in FIG. 14, A12-CAR-T can significantly kill T47D cells with positive ROR2 expression, but cannot kill MDA-MB-231 cells without ROR2 expression.

It can be seen from the above experiments that the development of ROR2-CAR T cell therapy technology uses the high-affinity ROR2 antibodies screened to further develop CAR-T cell therapy technology. There is currently no report on ROR2-CAR T cell therapy at home and abroad.

In the description of the specification, the description of the terms “an embodiment”, “some embodiments”, “example”, “specific example”, or “some examples” means that the specific features, structures, materials, or characteristics described in combination with the embodiment or example are included in at least one embodiment or example of the present disclosure. In this specification, the schematic representations of the above-mentioned terms are not necessarily directed to the same embodiment or example. Moreover, the described specific features, structures, materials or characteristics can be combined in any one or more embodiments or examples in a suitable manner. In addition, those skilled in the art can combine the different embodiments or examples and the features of the different embodiments or examples described in this specification without contradicting each other.

Although the embodiments of the present disclosure have been shown and described above, it can be understood that the above-mentioned embodiments are exemplary and should not be construed as limiting the present disclosure. Those of ordinary skill in the art can make changes, modifications, substitutions and modifications to the above-mentioned embodiments within the scope of the present disclosure.

What is claimed is:

1. An anti-ROR2 antibody or antigen-binding fragment, comprising at least one of the following:
(1) a light chain variable region with amino acid sequence shown in CSASSSVSYMHWYQ, IYDTSKLAS and CQQWSSNPPTFGAG, and a heavy chain variable region with amino acid sequence shown in YTITSYLMHWV, LEWIGYINPYNDGTKYNEKFKDKAT and CARSDVYYGVRFAYWGQG;
(2) a light chain variable region with amino acid sequence shown in CKASQNVGTNVAWFQ, IYLASYRYS and CQQYNSYPLTFGGG, and a heavy chain variable region with amino acid sequence shown in YTFTNYWIQWM, LEWIGEINPSNGRTDYNEKFKNRAT and CANYRPGYWGQG; and
(3) a light chain variable region with amino acid sequence shown in CSASSSISYMYWYQ, IYDTSILAS and CQQWSSYPFTFGSG, and a heavy chain variable region with amino acid sequence shown in YTFTSYLIHWV, LEWIGYINPYNDGTKYNEKFKDKAT and CARSDVYYGVRFAYWGQG;
compared with (1) to (3), the antibody or antigen-binding fragment has an amino acid sequence with at least one conservative amino acid substitution;
the anti-ROR2 antibody or antigen-binding fragment comprising at least one of the following:
(a) a light chain variable region with amino acid sequence shown in SEQ ID NO: 1 and a heavy chain variable region with amino acid sequence shown in SEQ ID NO: 2;
(b) a light chain variable region with amino acid sequence shown in SEQ ID NO: 3 and a heavy chain variable region with amino acid sequence shown in SEQ ID NO: 4; and
(c) a light chain variable region with amino acid sequence shown in SEQ ID NO: 5 and a heavy chain variable region with amino acid sequence shown in SEQ ID NO: 6;
compared with (a) to (c), the antibody or antigen-binding fragment has an amino acid sequence with at least one conservative amino acid substitution;
the anti-ROR2 antibody or antigen-binding fragment comprising at least one of the following:
a light chain with amino acid sequence shown in SEQ ID NO: 7 and a heavy chain with amino acid sequence shown in SEQ ID NO: 8;
a light chain with amino acid sequence shown in SEQ ID NO: 9 and a heavy chain with amino acid sequence shown in SEQ ID NO: 10; and
a light chain with amino acid sequence shown in SEQ ID NO: 11 and a heavy chain with amino acid sequence shown in SEQ ID NO: 12.

2. An isolated polynucleotide, wherein the polynucleotide encodes the antibody or antigen-binding fragment of claim 1, and comprises at least one of the following:
a light chain variable region nucleotide sequence shown in SEQ ID NO: 13 and a heavy chain variable region nucleotide sequence shown in SEQ ID NO: 14;
a light chain variable region nucleotide sequence shown in SEQ ID NO: 15 and a heavy chain variable region nucleotide sequence shown in SEQ ID NO: 16;
a light chain variable region nucleotide sequence shown in SEQ ID NO: 17 and a heavy chain variable region nucleotide sequence shown in SEQ ID NO: 18;
a light chain nucleotide sequence shown in SEQ ID NO: 19 and a heavy chain nucleotide sequence shown in SEQ ID NO: 20;
a light chain nucleotide sequence shown in SEQ ID NO: 21 and a heavy chain nucleotide sequence shown in SEQ ID NO: 22;
a light chain nucleotide sequence shown in SEQ ID NO: 23 and a heavy chain nucleotide sequence shown in SEQ ID NO: 24;
compared with any light chain variable region nucleotide sequence described above, the polynucleotide has a sequence of more than 90% homology, a sequence of more than 95% homology, a sequence of more than 98% homology, or a sequence of more than 99% homology;
compared with any heavy chain variable region nucleotide sequence described above, the polynucleotide has a sequence of more than 90% homology, a sequence of more than 95% homology, a sequence of more than 98% homology, or a sequence of more than 99% homology;
compared with any light chain nucleotide sequence described above, the polynucleotide has a sequence of more than 90% homology, a sequence of more than 95% homology, a sequence of more than 98% homology, or a sequence of more than 99% homology;
compared with any heavy chain nucleotide sequence described above, the polynucleotide has a sequence of more than 90% homology, a sequence of more than 95% homology, a sequence of more than 98% homology, or a sequence of more than 99% homology.

3. An expression vector, comprising:
the polynucleotide of claim 2; and
a control element operably connected to the polynucleotide, and for controlling expression of the polynucleotide in a host cell;
wherein the control element includes at least one of the following: a promoter, an enhancer and a terminator, and the host cell is a mammalian cell.

4. A recombinant cell, comprising the expression vector of claim 3.

5. A method for preparing an anti-ROR2 antibody or antigen-binding fragment, comprising culturing the recombinant cell of claim 4.

6. A use of the antibody or antigen-binding fragment of claim 1 in preparation of a medicine, wherein the medicine is for treatment of cancer.

7. A use of the antibody or antigen-binding fragment of claim 1 in preparation of a kit, wherein the kit is for diagnosis and detection of ROR2 antigen, and the kit is for immunoblotting, immunoprecipitation and/or ELISA detection.

8. A pharmaceutical composition, comprising: the antibody or antigen-binding fragment of claim 1 and a pharmaceutically acceptable carrier.

9. An anti-ROR2 chimeric antigen receptor, comprising: an extracellular domain, a transmembrane domain and an intracellular domain,
wherein the extracellular domain includes the antibody or antigen-binding fragment of claim 1, and the antibody or antigen-binding fragment is a single chain.

10. A CAR-T cell, wherein the CAR-T cell expresses the anti-ROR2 chimeric antigen receptor of claim 9.


SEQUENCE LISTING (24 sequences)

SEQ ID NO: 1 - protein, 106 aa, Artificial Sequence
  A12 light chain variable region
DIVLTQSPAIMSASPGEKVTMTCSASSSVSYMHWYQQKSGTSPKRWIYDTSKLASGVPARFSGSGSGTSYSLTISSMEAEDAAIYYCQQWSSNPPTFGAGTKLELK

SEQ ID NO: 2 - protein, 119 aa, Artificial Sequence
  A12 heavy chain variable region
QVQLQQSGPELVKPGASVRMSCKAAGYTITSYLMHWVKQRPGQDLEWIGYINPYNDGTKYNEKFKDKATLTSDKSSSTAYMELSSLTSEDSAVYYCARSDVYYGVRFAYWGQGTLVTVS

SEQ ID NO: 3 - protein, 108 aa, Artificial Sequence
  B22 light chain variable region
DIVMTQSQKFMSTSVGDRVSVTCKASQNVGTNVAWFQQKPGQSPKPLIYLASYRYSGVPDRFTGSGSGTDFTLTISNVQSEDLAEYFCQQYNSYPLTFGGGTNLEIKR

SEQ ID NO: 4 - protein, 114 aa, Artificial Sequence
  B22 heavy chain variable region
QVQLQQPGAALVKPGASVKLSCKASGYTFTNYWIQWMKQRPGQGLEWIGEINPSNGRTDYNEKFKNRATLTVDNSSTTAYMQLSSLTSEDSAVYYCANYRPGYWGQGTSVTVSS

SEQ ID NO: 5 - protein, 106 aa, Artificial Sequence
  B30 light chain variable region
DIVLTQSPVIMSASPGEKVTMTCSASSSISYMYWYQQKPGSSPRLLIYDTSILASGVPVRFSGSGSGTSYSLTISRMEAEDAATYYCQQWSSYPFTFGSGTKLEIK

SEQ ID NO: 6 - protein, 120 aa, Artificial Sequence
  B30 heavy chain variable region
QVQLQQSGPELVKPGASVRMSCKAAGYTFTSYLIHWVKQRPGQDLEWIGYINPYNDGTKYNEKFKDKATLTSDKSSSTAYMELSSLTSEDSAVYYCARSDVYYGVRFAYWGQGTLVTVSA

SEQ ID NO: 7 - protein, 213 aa, Artificial Sequence
  A12 light chain
DIVLTQSPAIMSASPGEKVTMTCSASSSVSYMHWYQQKSGTSPKRWIYDTSKLASGVPARFSGSGSGTSYSLTISSMEAEDAAIYYCQQWSSNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 449 aa, Artificial Sequence
  A12 heavy chain
QVQLQQSGPELVKPGASVRMSCKAAGYTITSYLMHWVKQRPGQDLEWIGYINPYNDGTKYNEKFKDKATLTSDKSSSTAYMELSSLTSEDSAVYYCARSDVYYGVRFAYWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, Artificial Sequence
  B22 light chain
DIVMTQSQKFMSTSVGDRVSVTCKASQNVGTNVAWFQQKPGQSPKPLIYLASYRYSGVPDRFTGSGSGTDFTLTISNVQSEDLAEYFCQQYNSYPLTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 444 aa, Artificial Sequence
  B22 heavy chain
QVQLQQPGAALVKPGASVKLSCKASGYTFTNYWIQWMKQRPGQGLEWIGEINPSNGRTDYNEKFKNRATLTVDNSSTTAYMQLSSLTSEDSAVYYCANYRPGYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 213 aa, Artificial Sequence
  B30 light chain
DIVLTQSPVIMSASPGEKVTMTCSASSSISYMYWYQQKPGSSPRLLIYDTSILASGVPVRFSGSGSGTSYSLTISRMEAEDAATYYCQQWSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 450 aa, Artificial Sequence
  B30 heavy chain
QVQLQQSGPELVKPGASVRMSCKAAGYTFTSYLIHWVKQRPGQDLEWIGYINPYNDGTKYNEKFKDKATLTSDKSSSTAYMELSSLTSEDSAVYYCARSDVYYGVRFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 318 bp, Artificial Sequence
  A12 light chain variable region coding sequence
gacatcgtgctgacccagtctccagccatcatgtctgctagccctggcgagaaagtgacaatgacctgctccgcctcctcctccgtgtcctacatgcactggtatcagcagaagtccggcacctctcctaagcggtggatctacgacacctccaagctggctagcggagtgcctgccagattttccggctctggctctggcacctcttactccctgaccatctcctccatggaagccgaggatgccgccatctactactgccagcagtggtctagcaaccctcctacctttggcgctggcaccaagctggaactgaag

SEQ ID NO: 14 - DNA, 357 bp, Artificial Sequence
  A12 heavy chain variable region coding sequence
caggttcagctgcagcagtctggacctgagctggttaagcctggtgcctccgtccggatgtcttgcaaggctgctggctacaccatcaccagctacctgatgcactgggtcaagcagaggccaggccaggacttggagtggatcggctacatcaacccctacaacgacggcaccaagtacaacgagaagttcaaggacaaggctaccctgacctccgacaagtcctcctccaccgcctacatggaactgtccagcctgacctctgaggactccgccgtgtactactgcgccagatccgatgtgtactatggcgtcagattcgcctactggggccagggcacactggtcacagtttct

SEQ ID NO: 15 - DNA, 318 bp, Artificial Sequence; identical to SEQ ID NO: 13
  B22 light chain variable region coding sequence

SEQ ID NO: 16 - DNA, 342 bp, Artificial Sequence
  B22 heavy chain variable region coding sequence
caggttcagttgcagcaacctggcgctgctctggttaagcctggcgcttctgtgaagctgtcctgcaaggcttccggctacaccttcaccaactattggatccagtggatgaagcagcggccaggccaaggcctggaatggatcggagagatcaacccttctaacggccggaccgactacaacgagaagttcaagaaccgggctaccctgaccgtggacaactcttctaccaccgcctacatgcagctgtccagcctgacctctgaggactccgccgtgtactactgcgccaactacagacctggctattggggccagggcacctctgtgacagtctcttct

SEQ ID NO: 17 - DNA, 321 bp, Artificial Sequence
  B30 light chain variable region coding sequence
gacatcgtgatgacccagagccagaaattcatgtccacctccgtgggcgacagagtgtccgtgacatgcaaggcctctcagaacgtgggcaccaacgtggcctggttccagcagaaacctggccagtctcctaagcctctgatctacctggcctcctaccggtactctggcgtgcccgatagattcaccggctctggatctggcaccgacttcaccctgaccatctccaacgtgcagtctgaggacctggccgagtacttctgccagcagtacaacagctaccctctgacctttggcggaggcaccaacctggaaatcaag

SEQ ID NO: 18 - DNA, 360 bp, Artificial Sequence
  B30 heavy chain variable region coding sequence
caggttcagctgcagcagtctggacctgagctggttaagcctggtgcctccgtccggatgtcttgcaaggctgccggctacaccttcaccagctacctgatccactgggtcaagcagaggccaggccaggacttggagtggatcggctacatcaacccctacaacgacggcaccaagtacaacgagaagttcaaggacaaggctaccctgacctccgacaagtcctcctccaccgcctacatggaactgtccagcctgacctctgaggactccgccgtgtactactgcgccagatccgatgtgtactatggcgtcagattcgcctactggggccagggcacactggtcacagtttctgct

SEQ ID NO: 19 - DNA, 639 bp, Artificial Sequence
  A12 light chain coding sequence
gacatcgtgctgacccagtctccagccatcatgtctgctagccctggcgagaaagtgacaatgacctgctccgcctcctcctccgtgtcctacatgcactggtatcagcagaagtccggcacctctcctaagcggtggatctacgacacctccaagctggctagcggagtgcctgccagattttccggctctggctctggcacctcttactccctgaccatctcctccatggaagccgaggatgccgccatctactactgccagcagtggtctagcaaccctcctacctttggcgctggcaccaagctggaactgaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgt

SEQ ID NO: 20 - DNA, 1347 bp, Artificial Sequence
  A12 heavy chain coding sequence
caggttcagctgcagcagtctggacctgagctggttaagcctggtgcctccgtccggatgtcttgcaaggctgctggctacaccatcaccagctacctgatgcactgggtcaagcagaggccaggccaggacttggagtggatcggctacatcaacccctacaacgacggcaccaagtacaacgagaagttcaaggacaaggctaccctgacctccgacaagtcctcctccaccgcctacatggaactgtccagcctgacctctgaggactccgccgtgtactactgcgccagatccgatgtgtactatggcgtcagattcgcctactggggccagggcacactggtcacagtttctgcctctacaaagggccctagtgtgttccctctggctcccagcagcaagtctacatctggcggaacagccgctctgggctgcctggtcaaggattactttcccgagcctgtgaccgtgtcctggaatagcggagcactgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggcaaa

SEQ ID NO: 21 - DNA, 642 bp, Artificial Sequence
  B22 light chain coding sequence
gacatcgtgatgacccagagccagaaattcatgtccacctccgtgggcgacagagtgtccgtgacatgcaaggcctctcagaacgtgggcaccaacgtggcctggttccagcagaaacctggccagtctcctaagcctctgatctacctggcctcctaccggtactctggcgtgcccgatagattcaccggctctggatctggcaccgacttcaccctgaccatctccaacgtgcagtctgaggacctggccgagtacttctgccagcagtacaacagctaccctctgacctttggcggaggcaccaacctggaaatcaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgt

SEQ ID NO: 22 - DNA, 1332 bp, Artificial Sequence
  B22 heavy chain coding sequence
caggttcagttgcagcaacctggcgctgctctggttaagcctggcgcttctgtgaagctgtcctgcaaggcttccggctacaccttcaccaactattggatccagtggatgaagcagcggccaggccaaggcctggaatggatcggagagatcaacccttctaacggccggaccgactacaacgagaagttcaagaaccgggctaccctgaccgtggacaactcttctaccaccgcctacatgcagctgtccagcctgacctctgaggactccgccgtgtactactgcgccaactacagacctggctattggggccagggcacctctgtgacagtctcttctgcctctacaaagggccctagtgtgttccctctggctcccagcagcaagtctacatctggcggaacagccgctctgggctgcctggtcaaggattactttcccgagcctgtgaccgtgtcctggaatagcggagcactgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggcaaa

SEQ ID NO: 23 - DNA, 639 bp, Artificial Sequence
  B30 light chain coding sequence
gacatcgtgctgacccagtctccagtgatcatgtccgcttctcccggcgagaaagtgacaatgacctgctccgcctcctccagcatctcctacatgtactggtatcagcagaagcccggctcctctcctcggctgctgatctacgatacctccatcctggcttccggcgtgccagtgcggttttctggttctggctctggcacctcctacagcctgaccatctccagaatggaagccgaggacgccgccacctactactgtcagcagtggtctagctaccccttcacctttggctccggcaccaagctggaaatcaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgt

SEQ ID NO: 24 - DNA, 1350 bp, Artificial Sequence
  B30 heavy chain coding sequence
caggttcagctgcagcagtctggacctgagctggttaagcctggtgcctccgtccggatgtcttgcaaggctgccggctacaccttcaccagctacctgatccactgggtcaagcagaggccaggccaggacttggagtggatcggctacatcaacccctacaacgacggcaccaagtacaacgagaagttcaaggacaaggctaccctgacctccgacaagtcctcctccaccgcctacatggaactgtccagcctgacctctgaggactccgccgtgtactactgcgccagatccgatgtgtactatggcgtcagattcgcctactggggccagggcacactggtcacagtttctgctgcctctacaaagggccctagtgtgttccctctggctcccagcagcaagtctacatctggcggaacagccgctctgggctgcctggtcaaggattactttcccgagcctgtgaccgtgtcctggaatagcggagcactgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggcaaa